(19) United States
(12) Patent Application Publication
(10) Pub. No.: US 2022/0338729 A1
(43) Pub. Date: Oct. 27, 2022

(54) SYSTEM AND METHOD FOR IDENTIFYING PRESBYOPIA

(71) Applicant: Johnson & Johnson Vision Care, Inc., Jacksonville, FL (US)

(72) Inventors: Greg Williby, Jacksonville, FL (US); Stuart Todd, Jacksonville, FL (US); Khaled Chehab, Jacksonville, FL (US); Lauren Westin, Jacksonville, FL (US); William Honey, Bath (GB); James Salisbury, Cambridge (GB); Glenn LeFaou, Ware (GB); Hannah Catton, Ely (GB)

(21) Appl. No.: 17/728,824

(22) Filed: Apr. 25, 2022

Related U.S. Application Data
(60) Provisional application No. 63/180,451, filed on Apr. 27, 2021.

(51) Int. Cl. A61B 3/032

(52) U.S. Cl. CPC A61B 3/032

(57) ABSTRACT

The present invention relates to new and improved systems and methods for identifying, evaluating and/or advising patients on the subject matter of presbyopia using a visual display in which presbyopes and non-presbyopes perceive different objects.

### CROSS REFERENCE TO RELATED APPLICATIONS

The present application claims priority to U.S. Provisional Patent Application No. 63/180,451 filed on Apr. 27, 2021.

### FIELD OF USE

The present invention relates generally to the field of human vision, and more specifically to new and improved systems and methods for identifying, evaluating and/or advising patients on the subject matter of presbyopia.

### BACKGROUND OF THE INVENTION

As a person ages their eyes are less able to accommodate or bend the natural lens to focus on objects that are relatively near to the observer. This condition is known as presbyopia. Similarly, people who have had their natural lens removed and an intraocular lens inserted as a replacement do not generally have the ability to accommodate.

Presbyopia can be corrected with eyeglasses, contact lenses or surgery. One approach for treating presbyopia is monovision, in which a single vision lens for correction of distance vision is used in the dominant eye and a single vision lens for correction of near vision is used in the non-dominant eye. Monovision typically results in a loss of stereopsis. Another method for treating presbyopia is the use of bifocal or multifocal contact lenses in both of the individual's eyes. Satisfactory corrections can be obtained with this method, but it typically results in a reduction of image contrast and resolution compared to monovision. Yet another method of treating presbyopia is modified monovision. This involves a bifocal or multifocal lens in the first eye and either a single vision lens or a bifocal or multifocal that is different than the first eye lens in the second eye. Modified monovision can require consideration of a large number of possible lenses in order to provide satisfactory lens performance.

Despite the various ways in which presbyopia can be treated, eye care practitioners face challenges in addressing presbyopia with patients and/or detecting early stages of presbyopia to best treat those patients. Most consumers or patients are not even aware of what presbyopia is or the associated symptoms and effects presbyopia and its progression can have such as blurry vision, eye strain and/or headaches when viewing objects at a close distance. When consumers or patients do not know of the problem, they cannot initiate discussions on the topic with eye care practitioners who may otherwise be able to provide a solution to help improve vision and better support their lifestyle by alleviating compensating behaviors often engaged to counteract presbyopic symptoms. Compounding this problem is the fact that many eye care professionals are hesitant themselves to raise the issue of presbyopia with patients too early or even at all, as the topic by necessity requires addressing aging and its associated maladies.

Given the barriers to communication around the subject matter of presbyopia, it would be beneficial to provide a system and method by which the effects of presbyopia can readily be demonstrated to even early presbyopes, thereby potentially identifying patients that may benefit from treatment options, and otherwise opening the door for communication on the subject of presbyopia.

### SUMMARY OF THE INVENTION

The present invention provides a method for identifying a potential presbyope, which may include presenting a visual display to an individual that has been generated using a grating pattern having a spatial frequency, at least a first portion of this display having a first duty cycle and at least a second portion having a second duty cycle different than the first duty cycle. The second portion is in the form of a predetermined shape, and its duty cycle is selected such that a presbyopic individual, when viewing at a preselected range of distances within which said individual cannot adequately accommodate, can see the predetermined shape, whereas the object is substantially camouflaged to a non-presbyopic individual. The method may further include the step of obtaining from the individual an indication of whether the individual can see the predetermined shape.

The spatial frequency may be greater than 2 cm⁻¹ and less than 6 cm⁻¹ and according to a preferred embodiment may be about 3.33 cm⁻¹. According to one embodiment, the second duty cycle may represent an increase in contrast of between 1 and 10 percent comparted to the first duty cycle, more preferably between 3 and 10 percent, and most preferably may be about 5 percent, and spatial frequency is greater than 2 cm⁻¹ and 6 cm⁻¹, preferably about 3.33 cm⁻¹.

According to yet another embodiment, the visual display further includes a second object at a location other than over the predetermined shape, wherein the second object does not include a grating pattern and is visible to both presbyopes and non-presbyopes.

In one embodiment, the grating pattern consists of lines that are straight, diagonal, triangular, wavy, circular or a combination thereof, and may consist of zigzag lines. The predetermined shape may be one or more alphanumeric characters, may be a living or inanimate object, or collection of objects, and/or may be the shape of a tear drop.

The visual display may be presented on a substantially flat backing material, which may be paper, a poster, or picture board. Alternatively, the visual display may be presented on an electronic screen, such as a computer screen, mobile device screen or tablet screen.

Also provided is an article incorporating a visual display for prompting a response from a patient, that may include a visual display presented on a substantially flat backing material, wherein the visual display is generated using a grating pattern having a spatial frequency at least a first portion of which has a first duty cycle and at least a second portion of which has a second duty cycle that is different from the first duty cycle, wherein the second portion is in the form of a predetermined shape. The second duty cycle may be selected such that a presbyopic individual when viewing at a preselected distance within which said individual cannot adequately accommodate can see the predetermined shape, whereas the object is substantially camouflaged to a non-presbyopic individual.

The article may further have a spatial frequency is greater than 2 cm⁻¹ and less than 6 cm⁻¹, and preferably may be about 3.33 cm⁻¹. The second duty cycle may represent an increase in contrast of between 1 and 10 percent compared to the first duty cycle, may further be between 3 and 7 percent, and may preferably by about 5 percent, when the spatial frequency is greater than 2 cm⁻¹ and 6 cm⁻¹.

In yet another embodiment, the visual display further includes a second object at a location other than over said predetermined shape, wherein the second object does not include a grating pattern and is visible to both presbyopes and non-presbyopes.

The grating pattern may consist of lines that are straight, diagonal, triangular, wavy, circular or a combination thereof, may consists of zigzag lines, and the predetermined shape may be one or more alphanumeric characters, that of a living or inanimate object, or collection of objects, and/or that of a tear drop.

The visual display may be presented on a substantially flat backing material, such as paper, a poster, or picture board.

The present invention also provides an article incorporating a visual display for prompting a response from a patient, including a visual display presented on an electronic screen, wherein said visual display is generated using a grating pattern having a spatial frequency at least a first portion of which has a first duty cycle and at least a second portion of which has a second duty cycle different than the first duty cycle, wherein said second portion is in the form of a predetermined shape. The second duty cycle is selected such that a presbyopic individual when viewing at a preselected distance within which said individual cannot adequately accommodate can see the predetermined shape, and whereas the object is substantially camouflaged to a non-presbyopic individual.

According to one embodiment, the spatial frequency is greater than 2 cm⁻¹ and less than 6 cm⁻¹, and preferably is about 3.33 cm⁻¹. The second duty cycle may represent an increase in contrast of between 1 and 10 percent compared to the first duty cycle, when the spatial frequency is greater than 2 cm⁻¹ and 6 cm⁻¹, preferably about 3.33 cm⁻¹. The second duty cycle more preferably may be an increase between 3 and 10 percent compared, and more preferably about 5 percent.

The electronic screen may be a computer monitor or television screen, or a mobile phone screen, tablet screen or other portable electronic device screen.

In yet another embodiment, the visual display further includes a second object at a location other than over said predetermined shape, wherein the second object does not include a grating pattern and is visible to both presbyopes and non-presbyopes.

In alternate embodiments, the grating pattern may consist of lines that are straight, diagonal, triangular, wavy, circular or a combination thereof, or zigzag lines. The predetermined shape may be in the form of one or more alphanumeric characters, a living or inanimate object, or collection of objects, or a tear drop.

A method is also provided for generating a visual display suitable for identifying a potential presbyope. The method includes selecting a grating pattern to be used in said visual display, selecting a spatial frequency for said grating pattern, selecting a first duty cycle for said grating pattern for at least a first portion of said visual display that is intended to be perceived by both a presbyope and a non-presbyope, and selecting a second duty cycle different from the first duty cycle for at least a second portion of the visual display. The second portion of the visual display is in the shape of one or more objects or alphanumeric characters, and the second duty cycle is selected such that a presbyope when viewing the visual display at a predetermined distance at which said presbyope cannot adequately accommodate can perceive the predetermined one or more objects or alphanumeric characters, and such that the at least one objects or alphanumeric characters are substantially camouflaged to a non-presbyope viewing the visual display at said predetermined distance.

The grating pattern may be a zigzag pattern, and the spatial frequency of the grating pattern may be between about 2 cm⁻¹ and 6 cm⁻¹, and more preferably about 3.33 cm⁻¹.

In one embodiment, the first duty cycle is approximately ⅓, and the second duty cycle may represent an increase in contrast of between 1 and 10 percent compared to the first duty cycle, may be between 3 and 10 percent, and may further be about 5 percent.

Also provided herein is an article incorporating a visual display for prompting a response from a patient including a visual display presented on a substantially flat backing material, wherein the visual display is generated using a grating pattern having a spatial frequency at least a first portion of which has first duty cycle and at least a second portion of which has a second duty cycle that is different from the first duty cycle, wherein the second portion is in the form of a predetermined shape. The second duty cycle is selected such that a presbyopic individual when viewing at a preselected distance within which said individual cannot adequately accommodate can see the predetermined shape, whereas the predetermined shape is substantially camouflaged to a non-presbyope. The visual display further includes one or more additional portions each in the form of a predetermined shape and each having a different duty cycle between the first and second duty cycles such that each of said one or more predetermined shapes has a varying degree of camouflage to a non-presbyope.

### BRIEF DESCRIPTION OF THE DRAWINGS

FIG. 1 illustrates various exemplary grating patterns;

FIG. 2 illustrates various spatial frequencies for the same zigzag grating pattern;

FIG. 2a illustrates an exemplary grating pattern with two different spatial frequencies with and without blurring;

FIG. 3 illustrates an exemplary visual display incorporating a grating pattern with a spatial frequency having different portions with first and second different duty cycles;

FIG. 4 illustrates the visual display incorporating the grating pattern of FIG. 3 with only a single duty cycle;

FIG. 5 illustrates to a non-presbyope how a presbyope views the visual display of FIG. 3;

FIG. 6 illustrates an exemplary visual display according to the present disclosure;

FIGS. 7 and 8 illustrate various exemplary visual displays according to the present disclosure;

FIG. 9 illustrates various grating patterns;

FIG. 10 illustrates a preferred embodiment of an exemplary grating pattern; and

FIG. 11 illustrates additional details of the grating pattern of FIG. 10.

### DETAILED DESCRIPTION OF THE INVENTION

The present disclosure provides a new system and method for identifying, evaluating and/or advising patients on the subject matter of presbyopia and otherwise facilitating conversations surrounding presbyopia as between an individual and an eye care practitioner.

Presbyopia is a visual condition that will affect almost the entire population at some point in their lifetime, with typical onset occurring as the individual's age reaches somewhere in the 40s or 50s of years. As noted above, most individuals are not aware of the condition and/or its associated symptoms and effects, and often engage in compensatory behavior as vision degrades rather than discuss the issue with an eye care practitioner. Since emerging presbyopia is most commonly an age-related condition, many eye care practitioners are uncomfortable initiating a conversation on the topic as also noted above.

The system and method described herein facilitates the identification of potential presbyope patients and the resulting communication with eye care practitioners on the topic, which, where appropriate, can facilitate identification of treatment options to improve vision and/or lifestyle for those patients.

Optical illusions of various types are well-known. A recent and well-known example is the “panda illusion”, which is artwork created by artist Ilja Klemencov with the famous panda logo from the World Wide Fund for Nature (WWF). This artwork is created using black and white zigzagging lines, which, when first viewed, appear as such. When viewed from afar, however, the artwork appears to reveal the image of a giant panda which was previously “hidden”. The image is created by manipulating the relative weighting of the alternating black and white lines in the zigzag pattern. The spatial frequency (overall dimensions of the repeating unit) is uniform, but at the location of the panda image the dark lines are slightly thicker. When viewed at a far enough distance, these thicker lines appear to the viewer in the form of an object as opposed to alternating, zigzagging lines.

The present invention leverages the same concept as the panda illusion but does so with a hidden object whose camouflage is specifically tailored to indicate the presence of presbyopia such that when presbyopes view the visual display at a close viewing distance (a distance at which they cannot accommodate or fully accommodate) they are able to see or perceive the hidden objects whereas non-presbyopes are not.

To work in this manner, the optical illusions incorporated herein are specifically chosen to account for visual effects created due to blur, which can occur due to both motion and/or imperfect focus of light on the retina. Due to their inability to accommodate to close distances, presbyopes have imperfect focus of the light (blur) when viewing at such distances. In other words, presbyopes have an inability to resolve fine detail (i.e., high spatial frequency content) at close distances compared to non-presbyopes, and that can cause a hidden object to be perceived to a presbyope whereas it is not perceived for a non-presbyope. The object is hidden or “camouflaged” to a non-presbyope when a grating pattern and the duty cycle(s) of that grating pattern is selectively chosen as described further below such that blur will cause the object to be visible to the optical system of a presbyope and not to the optical system of a non-presbyope.

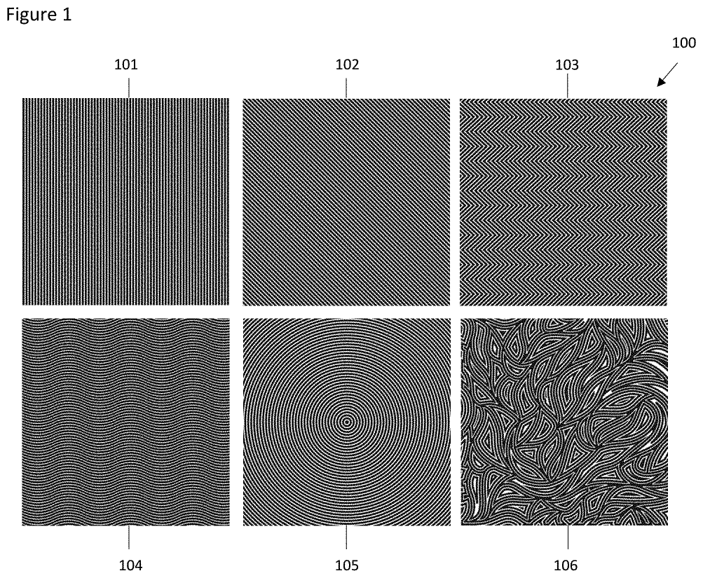

Camouflage is generally understood to mean the use of materials, colors, or illumination for concealment of an object. As used herein camouflage refers to patterned objects that are combined with other objects of interest in a scene in a manner such that the objects of interest are visible to the eye or otherwise perceived to exist under certain circumstances, but cannot be seen or perceived under different circumstances. One way to create a camouflaged object is to use grating patterns. Examples of a grating patterns 100 using lines are shown in FIG. 1, each of which incorporates alternating black and white lines in various formats such as straight lines 101, diagonal lines 102, triangular lines 103, wavy curves 104, circular curves 105 or more complex combination of lines or curves lines 106. Those skilled in the art readily recognize that grating patterns can be generated using elements other than lines, such as dots or pixels or other shapes and objects such as squares, diamonds, etc. Further, the grating patterns illustrated in FIG. 1 are in black and white, but relative shades of black and white or any other color can also be used.

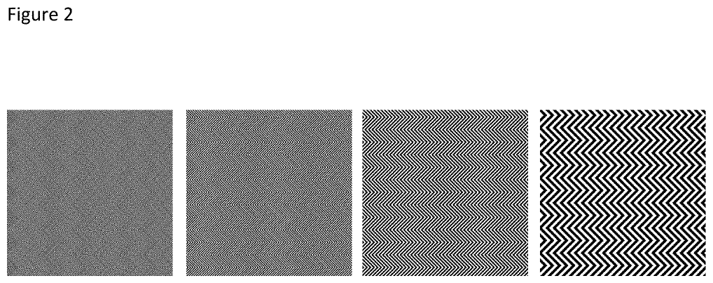

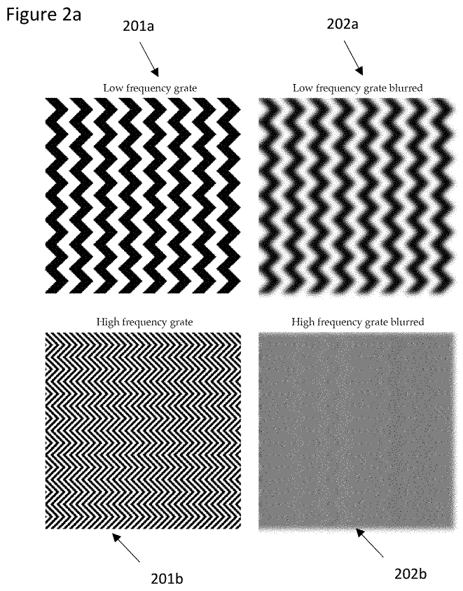

The spatial frequency of a grating pattern can be modified to alter its visual effect by increasing or decreasing the overall dimensions of the repeating pattern. FIG. 2 illustrates a zigzag grating pattern with different (decreasing) spatial frequencies, whereas FIG. 2a illustrates two exemplary lower and higher spatial frequencies 201a and 201b of a zigzag pattern, each with the same level of blurring applied (mimicking an exemplary presbyopic state) as shown in 202a and 202b respectively. In 202a, some detail of the grating pattern remains visible, whereas in 202b the grating pattern seems to substantially disappear. Blurring effectively results in an averaging of brightness to the individual across a limited spatial scale. An increase in blur results in widening this spatial scale. For the high frequency pattern, the spatial scale is large enough to average the brightness almost completely, whereas in the low frequency pattern differences in brightness can still be observed.

Visual perception of the average brightness across the limited spatial portion of an image can also be influenced by altering the relative thickness of the black or darker portions of the repeating pattern as compared to the white or lighter portions (the “duty cycle” as described further below in conjunction with FIG. 11). When the duty cycle of the image is high enough, an individual is less likely to perceive individual lines, pixels, objects or the like (i.e., individual alternating light/dark lines), but rather assigns an average brightness based on the relative luminance of the alternating lines, where thicker dark lines are perceived as overall dark areas and vice versa. If a presbyope is viewing the object, blur is also introduced by that individual's visual system and the duty cycle required for this effect to work is reduced, creating a window where presbyopes can perceive or see the object but non-presbyopes cannot. The object is hidden or camouflaged to the non-presbyope.

As noted above, blur impacts the efficacy of camouflage, and the effect of blur depends on the nature of the camouflage. If the camouflage is a high frequency pattern, blur reduces its efficacy as a camouflage, making it possible for a presbyope to spot an otherwise hidden object.

According to one embodiment, a visual display is provided that leverages camouflage technique(s) and principles of blur to create at least a first object that is visible at near distances to presbyopes who are unable to accommodate or accommodate fully at such a distance, but is not visible, or is less visible to non-presbyopes who are able to accommodate at such distances. The visual display may preferably further include a second object that does not incorporate camouflage techniques that is fully visible to both presbyopes and non-presbyopes at near and far distances.

In one example of the present invention, a visual display 301 is provided that includes a grating pattern 302 in the form of series of zigzag lines such as that shown in FIG. 3. Although figures provided herein are shown in black and white, those skilled in the art will readily understand that the same principles will apply whether black and white, or variations in shades of black and white, or shades of other colors. The visual display 301 may be in the form of a poster, a picture, an article in the form of a business card or other item that can be readily distributed, or other article on which the grating pattern is formed, printed or overlayed on, or may be on an electronic display such as a computer monitor, tablet, mobile phone screen or other electronic screen or display. The visual display may also be provided on external objects or advertising locations such as signage that is internal (i.e, stand-alone signage or signage affixed to walls).

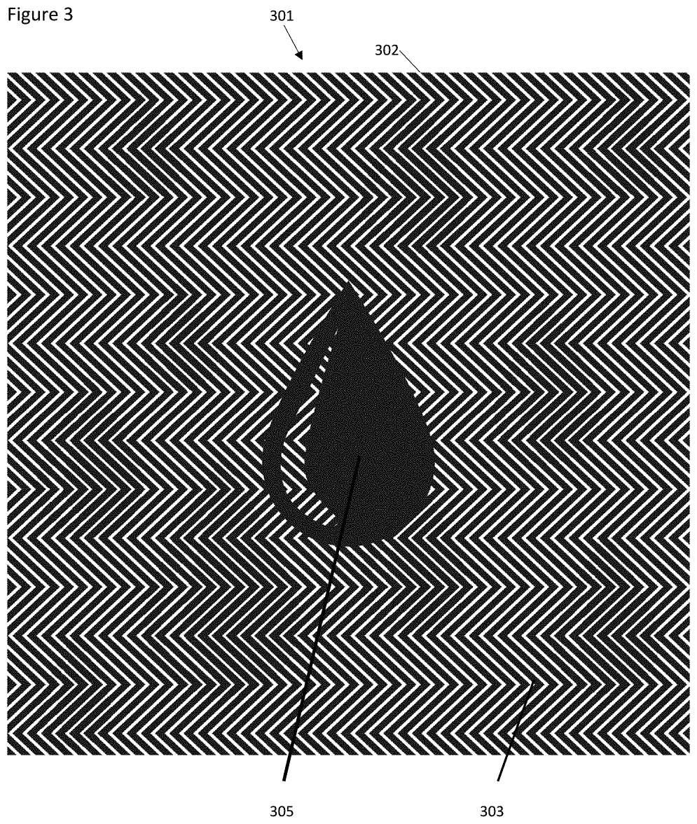

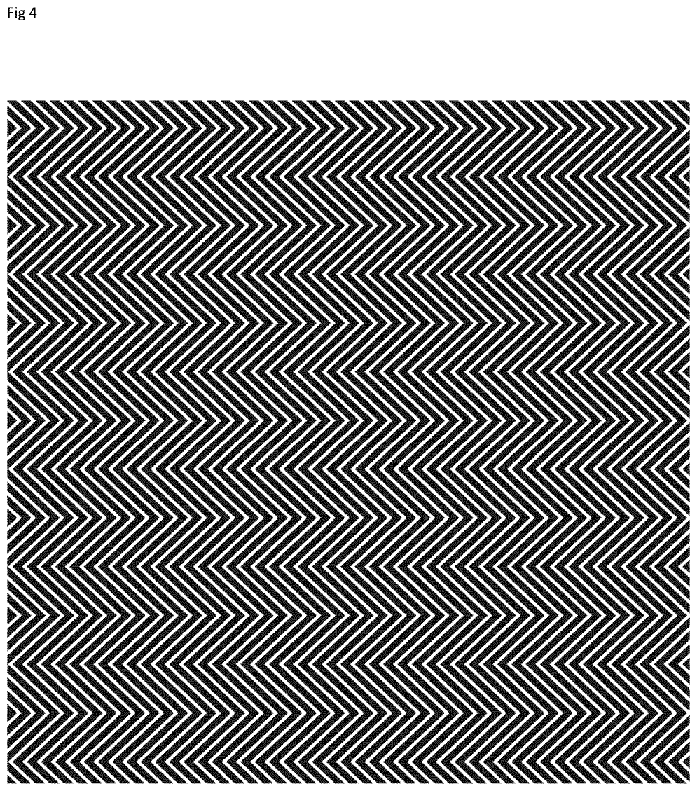

Within the grating pattern, at least one camouflaged or hidden object is included that has a duty cycle different from the remainder of the grating pattern. The different duty cycle is selected such that for individuals that can accommodate (non-presbyopes), the visual system will see the grating pattern as just that, a grating pattern (such as shown in FIG. 4). For those individuals that cannot accommodate (presbyopes), however, the added blur those individuals experience at near distances (defined as a distance closer than that at which they can accommodate) causes the visual system to perceive the object camouflaged within the grating pattern. Where the portion of the grating pattern having a different duty cycle is in the form of a shape such as a tear drop, the presbyope's visual system will see the image as it is shown in FIG. 5. For clarity, FIG. 5 is a different visual display designed only to illustrate to a non-presbyope how a presbyope would see FIG. 3.

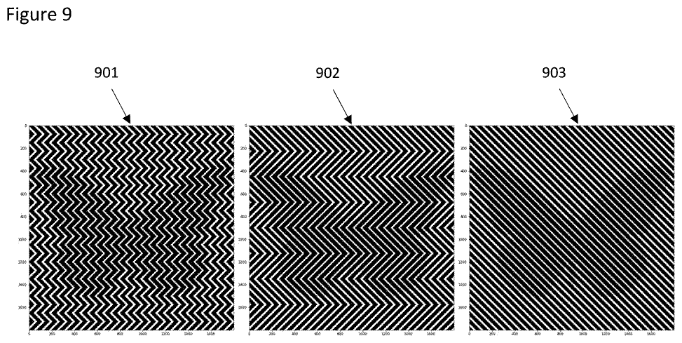

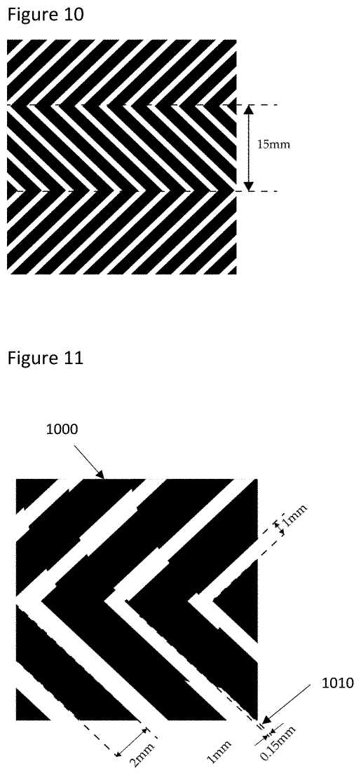

As noted, the principles described herein can be applied to develop tools that assist in the identification, evaluation and/or counseling of individuals with presbyopia, by generating a visual display having objects that can be observed by presbyopes, but are hidden or camouflaged to non-presbyopes. Several parameters should be taken into account when designing and generating an effective visual display for this purpose. One parameter is selecting an appropriate grating pattern for the visual display. Generally speaking, a grating pattern should be selected that averages locally to the same shade of grey across the entire display. As a general rule, grating patterns with a consistent duty cycle across ordered paths (such as horizontal line paths in 101, vertical line paths in 104 or radial paths from the center of 105) are preferable to more random patterns such as that shown in 106 of FIG. 1. Within consistent patterns, however, some level of detail to the grating pattern is preferable, as very simple grating patterns (alternating lines) do not capture the attention of the visual system and are, therefore, less effective at camouflage than more detailed patterns. This effect is illustrated in FIG. 9, where zigzag patterns 901, 902 provide better camouflage than straight lines 903 as the zigzag pattern captures the attention of the visual system, thereby better leaving the object unperceived. For the visual displays described further below, the grating pattern shown in FIG. 10 was selected as a preferred pattern, which has an overall grating pattern length of 15 mm with lines presented at 45 degrees relative to the horizontal axis.

Once the grating pattern is selected, the details of the spatial pattern and the duty cycle for the grating pattern, and how it varies in the areas of the visual display including the hidden or camouflaged object, must be chosen. These details will likely depend on the grating pattern that is chosen. For the embodiments described below, the details of the preferred grating pattern of FIG. 10 are shown in more detail in FIG. 11. For the portion of the visual display without a hidden image 1000, the preferable width of the alternating white lines is 1 mm and the preferable width of the alternative black lines is 2 mm such that the period of the repeating pattern is 3 mm (2 mm+1 mm) and the spatial frequency is 3.33 cm⁻¹ (inverse of the period). As noted above, the spatial frequency of the grating is critical to achieving the desired effect. If too high, the object is very easy to see, whereas if selected too low, it will be difficult to see even for an individual with very blurry vision. This, of course, is also dependent on the distance at which the visual display is intended to be viewed. The preferred spatial frequency of 3.33 cm⁻¹ described above was selected for visual displays intended to be viewed at 40 cm, although spatial frequencies within the range of greater than about 2 cm⁻¹ to less than about 6 cm⁻¹ may be suitable.

Once the spatial frequency is determined the duty cycle can be optimized. The duty cycle is the proportion of the white line over the period (i.e., 1 mm/3 mm). For the portion of the visual display incorporating a hidden object 1010, the duty cycle is manipulated so as to change the visual contrast of the repeating lines. This is done by increasing the width of the dark lines by 0.15 mm as illustrated, which simultaneously reduces the width of the white lines by 0.15 mm. The percent contrast change is 0.15 mm over the 3 mm period, or 5% increase in the black portion of the alternating pattern. If the contrast change is too low, the underlying image is difficult to perceive even when severely blurred (i.e., for a severe presbyope). If is it too high, the image is readily perceived even with no blurring. For the embodiments including the specific grating pattern and duty cycle described herein, it has been discovered that 5% contrast is close to optimal, 3 to 7% may be suitable, and 1% is too low and 10% too high. Optimal ranges will ultimately depend on the chosen grating pattern, spatial frequency and duty cycle, as well as chosen colors and/or shades used.

Although the pattern and details described above are a preferred embodiment, those skilled in the art will readily understand that the optimal parameters for any visual display relate to the specifics of human perception, both the physiology of the eye and the details of the image processing done in the brain, and thus are ultimately optimized by at least some level of real world trails with patients and/or designers using aids to mimic presbyopia.

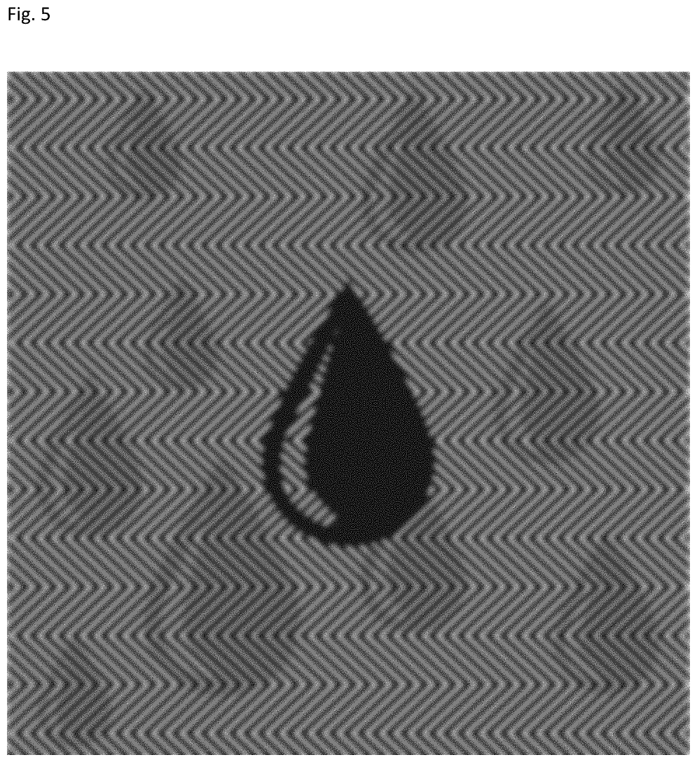

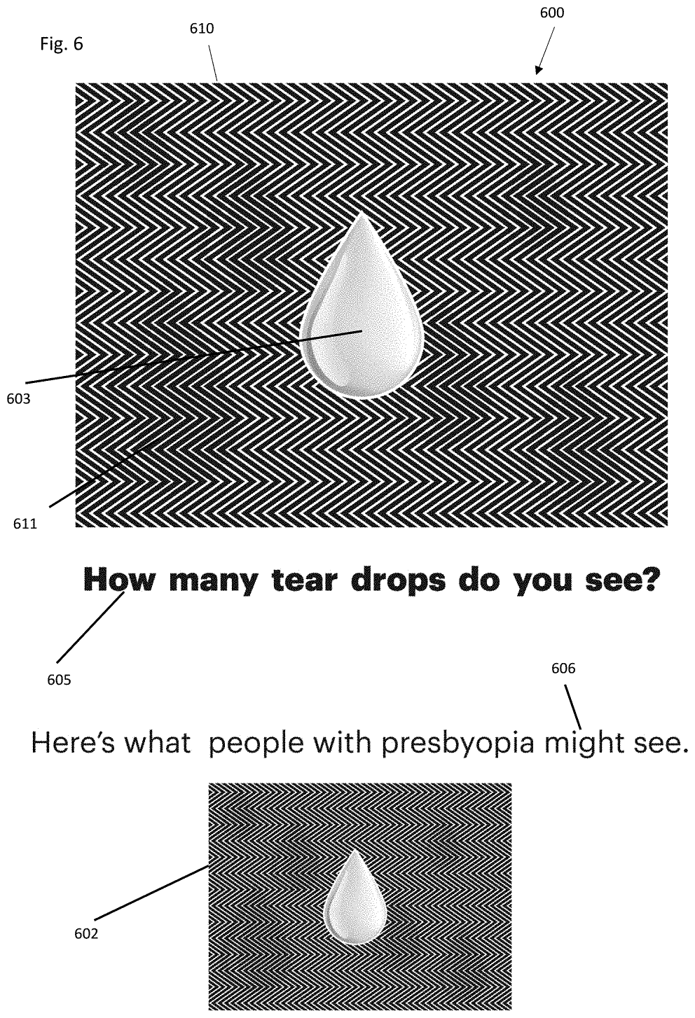

The visual display 600 (FIG. 6) may also preferably further include over the grating pattern 610 at least one object that does not incorporate a grating pattern, such as the tear drop 305 shown in FIG. 3 or tear drop 603 shown in FIG. 6. This object is an ordinary solid (non-camouflaged) object and will be viewed accordingly by presbyopes and non-presbyopes in the standard fashion. Finally, the visual display may include a separate portion 602 that is intended to display to a non-presbyope how the visual system of a presbyope would view the first portion of the visual display in a manner similar to that described above in connection with FIG. 3. Additional text elements 605, 606 may also be included that draw a patient's attention to the objects, invoke a response from the patient and/or are designed to stimulate conversation around the visual display.

A method for discussing presbyopia or identifying a presbyope can include presenting a visual display (i.e, 600) to an individual, where the visual display includes a grating pattern 610, with at least a portion 611 of the grating pattern having a different duty cycle, where that portion is a predefined shape such as an object, picture, alpha-numeric character or the like (herein after collectively referred to as “hidden object”). The different duty cycle is specifically selected to be a value within a range such that blur induced for a presbyopic patient viewing the visual object at a distance at which that patient cannot accommodate causes the presbyopic patient to see the hidden object whereas a non-presbyopic patient that can accommodate and does not have induced blur does not readily see the hidden object. Following presentation of the visual display to the patient, the eye care practitioner or other presenter will obtain from the patient (which may be following prompting or other questioning) an indication of whether the patient can or cannot see the hidden object. Based on this response, a conversation around presbyopia can be facilitated as can discussions around treatment options if necessary.

As noted above, if a second portion is present on the visual display a non-presbyope individual can be instructed to view the second portion to readily understand the different visual perception of presbyopes.

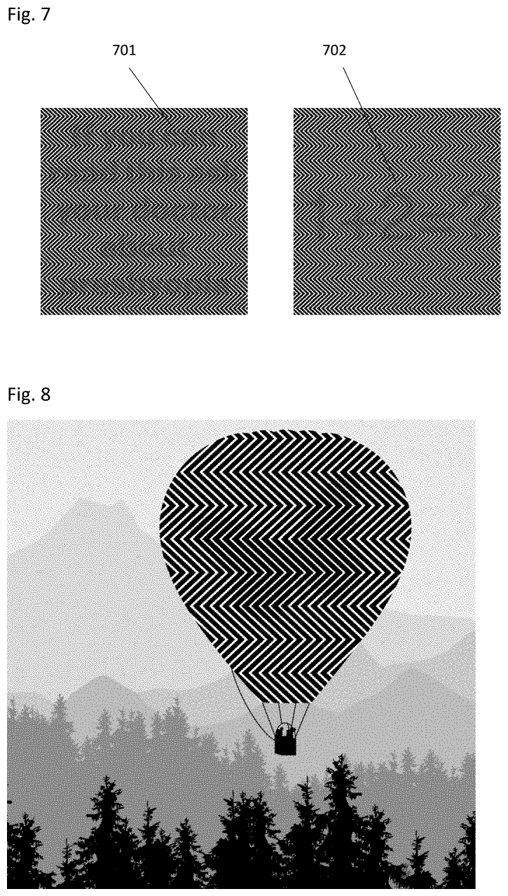

Although tear drop objects and hidden objects have been used throughout as exemplary embodiments, those skilled in the art readily understand that a broad variety of objects or collections of objects can be created leveraging the teachings of the present disclosure. For example, FIG. 7 illustrates examples using alphanumeric characters 701, 702 as hidden objects, and FIG. 8 illustrates an example using a picture or illustration 801. For the purposes of non-presbyopes that may be reading this specification, FIG. 7 and FIG. 8 have been generated solely to illustrate hidden objects that could be incorporated, and the specific spatial frequencies of the grating patterns described above have not been applied to enable presbyopes and non-presbyopes alike to see and thereby understand these examples. As noted above, color may also be used in any grating pattern to create a visual object and further designs may be added to the visual object to increase viewer interest and ultimately awareness to presbyopia.

In addition to the embodiments described above, visual displays could also be constructed including one or more additional portions each having one or more objects generated using the same grating pattern and spatial frequency, but with a series of different and incrementally greater duty cycles between that of the first and second duty cycles, which will result in varying degrees of camouflage of the objects on these different portions of the visual display. The varying degrees of camouflage may assist a health care practitioner in identifying or assessing the magnitude of presbyopia in a specific individual based on which of the several portions can be perceived by that individual. In one embodiment, the numbers one through nine may be presented with one generated using the first duty cycle and nine generated using the second. The numbers two through eight would each be generated with incrementally increasing duty cycles between the first and second duty cycle. Having the viewer identify which numbers he or she can perceive could provide additional insight to a health care practitioner as to the magnitude of that viewer's presbyopia.

Although illustrative embodiments have been described herein with reference to the accompanying drawings, it is to be understood that the invention is not limited to those precise embodiments and that various other changes and modifications may be affected herein by one skilled in the art without departing from the scope or spirit of the invention, which is limited only by the claims herein.

What is claimed is:

1. A method for identifying a potential presbyope comprising the steps of:
presenting a visual display to an individual that has been generated using a grating pattern having a spatial frequency, at least a first portion of which has a first duty cycle and at least a second portion of which has a second duty cycle different than the first duty cycle, wherein said second portion is in the form of a predetermined shape,
wherein the second duty cycle is selected such that a presbyopic individual when viewing at a preselected range of distances within which said individual cannot adequately accommodate can see the predetermined shape, whereas the object is substantially camouflaged to a non-presbyopic individual; and.
obtaining from said individual an indication of whether said individual can see said predetermined shape.

2. The method according to claim 1, wherein the spatial frequency is greater than 2 cm⁻¹ and less than 6 cm⁻¹

3. The method according to claim 2, wherein the spatial frequency is about 3.33 cm⁻¹.

4. The method according to claim 1, wherein the second duty cycle represents an increase in contrast of between 1 and 10 percent compared to the first duty cycle, and wherein the spatial frequency is greater than 2 cm⁻¹ and 6 cm⁻¹.

5. The method according to claim 4, wherein the spatial frequency is about 3.33 cm⁻¹.

6. The method according to claim 5, wherein the second duty cycle represents an increase in contrast of between 3 and 10 percent compared to the first duty cycle.

7. The method according to claim 6, wherein the increase in contrast is about 5 percent.

8. The method according to claim 1, wherein said visual display further includes a second object at a location other than over said predetermined shape, wherein the second object does not include a grating pattern and is visible to both presbyopes and non-presbyopes.

9. The method according to claim 1, wherein the grating pattern consists of lines that are straight, diagonal, triangular, wavy, circular or a combination thereof.

10. The method according to claim 9, wherein the grating pattern consists of zigzag lines.

11. The method according to claim 1, wherein the predetermined shape is that of one or more alphanumeric characters.

12. The method according to claim 1, wherein the predetermined shape is that of a living or inanimate object, or collection of objects.

13. The method according to claim 12, wherein the predetermined shape is that of a tear drop.

14. The method according to claim 1, wherein the visual display is presented on a substantially flat backing material.

15. The method according to claim 14, wherein the substantially flat backing material is paper, a poster, poster board or picture board.

16. The method according to claim 15, wherein the substantially flat backing material is poster board.

17. The method according to claim 1, wherein the visual display is presented on an electronic screen.

18. The method according to claim 17, wherein the electronic screen is a computer screen, mobile device screen or tablet screen.

19. An article incorporating a visual display for prompting a response from a patient, comprising:
a visual display presented on a substantially flat backing material, wherein said visual display is generated using a grating pattern having a spatial frequency at least a first portion of which has a first duty cycle and at least a second portion of which has a second duty cycle that is different from the first duty cycle, wherein the second portion is in the form of a predetermined shape,
wherein the second duty cycle is selected such that a presbyopic individual when viewing at a preselected distance within which said individual cannot adequately accommodate can see the predetermined shape, whereas the object is substantially camouflaged to a non-presbyopic individual.

20. The article according to claim 19, wherein the spatial frequency is greater than 2 cm⁻¹ and less than 6 cm⁻¹

21. The article according to claim 20, wherein the spatial frequency is about 3.33 cm⁻¹.

22. The article according to claim 19, wherein the second duty cycle represents an increase in contrast of between 3 and 10 percent compared to the first duty cycle, and wherein the spatial frequency is greater than 2 cm⁻¹ and 6 cm⁻¹.

23. The article according to claim 22, wherein the spatial frequency is about 3.33 cm⁻¹.

24. The article according to claim 23, wherein the second duty cycle represents an increase in contrast of between 3 and 10 percent compared to the first duty cycle.

25. The article according to claim 24, wherein the increase in contrast is about 5 percent.

26. The article according to claim 19, wherein said visual display further includes a second object at a location other than over said predetermined shape, wherein the second object does not include a grating pattern and is visible to both presbyopes and non-presbyopes.

27. The article according to claim 19, wherein the grating pattern consists of lines that are straight, diagonal, triangular, wavy, circular or a combination thereof.

28. The article according to claim 27, wherein the grating pattern consists of zigzag lines.

29. The article according to claim 19, wherein the predetermined shape is that of one or more alphanumeric characters.

30. The article according to claim 19, wherein the predetermined shape is that of a living or inanimate object, or collection of objects.

31. The article according to claim 30, wherein the predetermined shape is that of a tear drop.

32. The article according to claim 19, wherein the visual display is presented on a substantially flat backing material.

33. The article according to claim 32, wherein the substantially flat backing material is paper, a poster, poster board or picture board.

34. The article according to claim 33, wherein the substantially flat backing material is poster board.

35. An article incorporating a visual display for prompting a response from a patient, comprising:
a visual display presented on an electronic screen, wherein said visual display is generated using a grating pattern having a spatial frequency at least a first portion of which has a first duty cycle and at least a second portion of which has a second duty cycle different than the first duty cycle, wherein said second portion is in the form of a predetermined shape,
wherein the second duty cycle is selected such that a presbyopic individual when viewing at a preselected distance within which said individual cannot adequately accommodate can see the predetermined shape, and whereas the object is substantially camouflaged to a non-presbyopic individual.

36. The article according to claim 35, wherein the spatial frequency is greater than 2 cm⁻¹ and less than 6 cm⁻¹

37. The article according to claim 36, wherein the spatial frequency is about 3.33 cm⁻¹.

38. The article according to claim 35, wherein the second duty cycle represents an increase in contrast of between 1 and 10 percent compared to the first duty cycle, and wherein the spatial frequency is greater than 2 cm⁻¹ and less than 6 cm⁻¹.

39. The article according to claim 38, wherein the spatial frequency is about 3.33 cm⁻¹.

40. The article according to claim 39, wherein the second duty cycle represents an increase in contrast of between 3 and 10 percent compared to the first duty cycle.

41. The article according to claim 40, wherein the increase in contrast is about 5 percent.

42. The article according to claim 35, wherein the electronic screen is a computer monitor or television screen.

43. The article according to claim 35, wherein the electronic screen is a mobile phone screen, tablet screen or other portable electronic device screen.

44. The article according to claim 35, wherein said visual object further includes a second object at a location other than over said predetermined shape, wherein the second object does not include a grating pattern and is visible to both presbyopes and non-presbyopes.

45. The article according to claim 35, wherein the grating pattern consists of lines that are straight, diagonal, triangular, wavy, circular or a combination thereof.

46. The article according to claim 45, wherein the grating pattern consists of zigzag lines.

47. The article according to claim 35, wherein the predetermined shape is in the form of one or more alphanumeric characters.

48. The article according to claim 35, wherein the predetermined shape is in the form of a living or inanimate object, or collection of objects.

49. The article according to claim 48, wherein the predetermined shape is in the form of a tear drop.

50. A method for generating a visual display suitable for identifying a potential presbyope, comprising:
selecting a grating pattern to be used in said visual display;
selecting a spatial frequency for said grating pattern;
selecting a first duty cycle for said grating pattern for at least a first portion of said visual display that is intended to be perceived by both a presbyope and a non-presbyope;
selecting a second duty cycle different from the first duty cycle for at least a second portion of the visual display, wherein the second portion of the visual display is in the shape of one or more objects or alphanumeric characters, and wherein the second duty cycle is selected such that a presbyope when viewing the visual display at a predetermined distance at which said presbyope cannot adequately accommodate can perceive the predetermined one or more objects or alphanumeric characters, and such that the at least one objects or alphanumeric characters are substantially camouflaged to a non-presbyope viewing the visual display at said predetermined distance.

51. The method according to claim 50, wherein the grating pattern is a zigzag pattern.

52. The method according to claim 51, wherein the spatial frequency of the grating pattern is between about 2 cm⁻¹ and 6 cm⁻¹.

53. The method according to claim 52, wherein the spatial frequency of the grating pattern is about 3.33 cm⁻¹.

54. The method according to claim 53, wherein the first duty cycle is approximately ⅓.

55. The method according to claim 54, wherein the second duty cycle represents an increase in contrast of between 1 and 10 percent compared to the first duty cycle.

56. The method according to claim 55, wherein the second duty cycle represents an increase in contrast of between 3 and 10 percent compared to the first duty cycle.

57. The method according to claim 56, wherein the second duty cycle represents an increase in contrast of about 5 percent compared to the first duty cycle.

58. An article incorporating a visual display for prompting a response from a patient, comprising:
a visual display presented on a substantially flat backing material, wherein said visual display is generated using a grating pattern having a spatial frequency at least a first portion of which has first duty cycle and at least a second portion of which has a second duty cycle that is different from the first duty cycle, wherein the second portion is in the form of a predetermined shape,
wherein the second duty cycle is selected such that a presbyopic individual when viewing at a preselected distance within which said individual cannot adequately accommodate can see the predetermined shape, whereas the predetermined shape is substantially camouflaged to a non-presbyope, and
wherein the visual display further includes one or more additional portions each in the form of a predetermined shape and each having a different duty cycle between the first and second duty cycles such that each of said one or more predetermined shapes has a varying degree of camouflage to a non-presbyope.
